SEQUENCE LISTING (5 sequences)

SEQ ID NO: 1 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 2 - protein, 1025 aa, Homo sapiens
MVAMAEAEAGVAVEVRGLPPAVPDELLTLYFENRRRSGGGPVLSWQRLGCGGVLTFREPADAERVLAQADHELHGAQLSLRPAPPRAPARLLLQGLPPGTTPQRLEQHVQALLRASGLPVQPCCALASPRPDRALVQLPKPLSEADVRVLEEQAQNLGLEGTLVSLARVPQARAVRVVGDGASVDLLLLELYLENERRSGGGPLEDLQRLPGPLGTVASFQQWQVAERVLQQEHRLQGSELSLVPHYDILEPEELAENTSGGDHPSTQGPRATKHALLRTGGLVTALQGAGTVTMGSGEEPGQSGASLRTGPMVQGRGIMTTGSGQEPGQSGTSLRTGPMGSLGQAEQVSSMPMGSLEHEGLVSLRPVGLQEQEGPMSLGPVGSAGPVETSKGLLGQEGLVEIAMDSPEQEGLVGPMEITMGSLEKAGPVSPGCVKLAGQEGLVEMVLLMEPGAMRFLQLYHEDLLAGLGDVALLPLEGPDMTGFRLCGAQASCQAAEEFLRSLLGSISCHVLCLEHPGSARFLLGPEGQHLLQGLEAQFQCVFGTERLATATLDTGLEEVDPTEALPVLPGNAHTLWTPDSTGGDQEDVSLEEVRELLATLEGLDLDGEDWLPRELEEEGPQEQPEEEVTPGHEEEEPVAPSTVAPRWLEEEAALQLALHRSLEPQGQVAEQEEAAALRQALTLSLLEQPPLEAEEPPDGGTDGKAQLVVHSAFEQDVEELDRALRAALEVHVQEETVGPWRRTLPAELRARLERCHGVSVALRGDCTILRGFGAHPARAARHLVALLAGPWDQSLAFPLAASGPTLAGQTLKGPWNNLERLAENTGEFQEVVRAFYDTLDAARSSIRVVRVERVSHPLLQQQYELYRERLLQRCERRPVEQVLYHGTTAPAVPDICAHGFNRSFCGRNATVYGKGVYFARRASLSVQDRYSPPNADGHKAVFVARVLTGDYGQGRRGLRAPPLRGPGHVLLRYDSAVDCICQPSIFVIFHDTQALPTHLITCEHVPRASPDDPSGLPGRSPDT

SEQ ID NO: 3 - protein, 1638 aa, Homo sapiens
MLILLVENISGLSNDDFQVEIIRDFDVAVVTFQKHIDTIRFVDDCTKHHSIKQLQLSPRLLEVTNTIRVENLPPGADDYSLKLFFENPYNGGGRVANVEYFPEESSALIEFFDRKVLDTIMATKLDFNKMPLSVFPYYASLGTALYGKEKPLIKLPAPFEESLDLPLWKFLQKKNHLIEEINDEMRRCHCELTWSQLSGKVTIRPAATLVNEGRPRIKTWQADTSTTLSSIRSKYKVNPIKVDPTMWDTIKNDVKDDRILIEFDTLKEMVILAGKSEDVQSIEVQVRELIESTTQKIKREEQSLKEKMIISPGRYFLLCHSSLLDHLLTECPEIEICYDRVTQHLCLKGPSADVYKAKCEIQEKVYTMAQKNIQVSPEIFQFLQQVNWKEFSKCLFIAQKILALYELEGTTVLLTSCSSEALLEAEKQMLSALNYKRIEVENKEVLHGKKWKGLTHNLLKKQNSSPNTVIINELTSETTAEVIITGCVKEVNETYKLLFNFVEQNMKIERLVEVKPSLVIDYLKTEKKLFWPKIKKVNVQVSFNPENKQKGILLTGSKTEVLKAVDIVKQVWDSVCVKSVHTDKPGAKQFFQDKARFYQSEIKRLFGCYIELQENEVMKEGGSPAGQKCFSRTVLAPGVVLIVQQGDLARLPVDVVVNASNEDLKHYGGLAAALSKAAGPELQADCDQIVKREGRLLPGNATISKAGKLPYHHVIHAVGPRWSGYEAPRCVYLLRRAVQLSLCLAEKYKYRSIAIPAISSGVFGFPLGRCVETIVSAIKENFQFKKDGHCLKEIYLVDVSEKTVEAFAEAVKTVFKATLPDTAAPPGLPPAAAGPGKTSWEKGSLVSPGGLQMLLVKEGVQNAKTDVVVNSVPLDLVLSRGPLSKSLLEKAGPELQEELDTVGQGVAVSMGTVLKTSSWNLDCRYVLHVVAPEWRNGSTSSLKIMEDIIRECMEITESLSLKSIAFPAIGTGNLGFPKNIFAELIISEVFKFSSKNQLKTLQEVHFLLHPSDHENIQAFSDEFARRANGNLVSDKIPKAKDTQGFYGTVSSPDSGVYEMKIGSIIFQVASGDITKEEADVIVNSTSNSFNLKAGVSKAILECAGQNVERECSQQAQQRKNDYIITGGGFLRCKNIIHVIGGNDVKSSVSSVLQECEKKNYSSICLPAIGTGNAKQHPDKVAEAIIDAIEDFVQKGSAQSVKKVKVVIFLPQVLDVFYANMKKREGTQLSSQQSVMSKLASFLGFSKQSPQKKNHLVLEKKTESATFRVCGENVTCVEYAISWLQDLIEKEQCPYTSEDECIKDFDEKEYQELNELQKKLNINISLDHKRPLIKVLGISRDVMQARDEIEAMIKRVRLAKEQESRADCISEFIEWQYNDNNTSHCFNKMTNLKLEDARREKKKTVDVKINHRHYTVNLNTYTATDTKGHSLSVQRLTKSKVDIPAHWSDMKQQNFCVVELLPSDPEYNTVASKFNQTCSHFRIEKIERIQNPDLWNSYQAKKKTMDAKNGQTMNEKQLFHGTDAGSVPHVNRNGFNRSYAGKNAVAYGKGTYFAVNANYSANDTYSRPDANGRKHVYYVRVLTGIYTHGNHSLIVPPSKNPQNPTDLYDTVTDNVHHPSLFVAFYDYQAYPEYLITFRK

SEQ ID NO: 4 - protein, 656 aa, Homo sapiens [sequence omitted]

SEQ ID NO: 5 - protein, 323 aa, Homo sapiens
MQPSGWAAAREAAGRDMLAADLRCSLFASALQSYKRDSVLRPFPASYARGDCKDFEALLADASKLPNLKELLQSSGDNHKRAWDLVSWILSSKVLTIHSAGKAEFEKIQKLTGAPHTPVPAPDFLFEIEYFDPANAKFYETKGERDLIYAFHGSRLENFHSIIHNGLHCHLNKTSLFGEGTYLTSDLSLALIYSPHGHGWQHSLLGPILSCVAVCEVIDHPDVKCQTKKKDSKEIDRRRARIKHSEGGDIPPKYFVVTNNQLLRVKYLLVYSQKPPKSRASSQLSWFSSHWFTVMISLYLLLLLIVSVINSSAFQHFWNRAKR

(19) United States
(12) Patent Application Publication
(10) Pub. No.: US 2011/0070595 A1
(43) Pub. Date: Mar. 24, 2011

(54) METHODS FOR THE IDENTIFICATION OF PARP INTERACTING MOLECULES AND FOR PURIFICATION OF PARP PROTEINS

(75) Inventors: Gerard Drewes, Heidelberg (DE); Valerie Reader, Cambridge (GB)

(73) Assignee: Cellzome AG

(21) Appl. No.: 12/993,071

(22) PCT Filed: May 14, 2009
(86) PCT No.: PCT/EP09/03426

§ 371 Date: Nov. 16, 2010

(30) Foreign Application Priority Data
May 16, 2008 (EP) 08009128.3

(51) Int. Cl. G01N 33/573; C07D 471/04; C12N 9/10; C08B 37/00

(52) U.S. Cl. 435/7.4; 546/118; 435/193; 536/55.1

(57) ABSTRACT

The present invention relates to immobilization compounds and methods useful for the identification of PARP interacting compounds or for the purification or identification of PARP proteins.

The present invention relates to immobilization compounds and methods useful for the identification of PARP interacting molecules and for the purification of PARP proteins. Furthermore, the present invention relates to pharmaceutical compositions comprising said interacting molecules e.g. for the treatment of cancer, metabolic diseases or autoimmune/inflammatory disorders and to methods for the diagnosis of cancer.

Poly(ADP-ribose) polymerases (PARPs) constitute a family of cell signalling enzymes present in eukaryotes which catalyse poly(ADP-ribosylation) of DNA-binding proteins. PARPS are also known as poly(ADP-ribose) synthethases or poly(ADP-ribose) transferases (pARTs). These enzymes play an important role in the immediate cellular response to DNA damage. In response to DNA damage induced by ionizing radiation, oxidative stress and DNA-binding anti-tumour drugs, PARPs add ADP-ribose units to carboxlate groups of aspartic and glutamic residues of target proteins. This poly(ADP-ribosylation) is a posttranslational modification that triggers the inactivation of the acceptor protein through the attachment of a complex branched polymer of ADP-ribose units (Schreiber et al., 2006. Nature Reviews Cell Biology 7, 517-528).

ADP ribosylation is a posttranslational protein modification in which the ADP-ribose moiety is transferred from NAD onto specific amino acid side chains of target proteins (Schreiber et al., 2006. Nature Reviews Cell Biology 7, 517-528).

PARP1 is the first characterized and the best known member of the PARP family. This protein, encoded by the ADPRT gene, is a highly conserved chromatin bound enzyme which binds nicked DNA and mediates protection against DNA damage. In response to extensive DNA damage, PARP1 is overactivated and induces depletion of cellular NAD+ and ATP leading to cell dysfunction or cell death. Overactivation of PARP1 is involved in the pathogenesis of several diseases including stroke, myocardial infarctation, diabetes, neurodegenerative disorders and inflammatory diseases.

Targeting PARPs with small molecule inhibitors has been proposed as a promising approach for the cancer therapy (Haince et al., 2005. Trends Mol. Med. 11(10):456-63). Several PARP inhibitors have been reported in the literature. However, the first generation of PARP inhibitors is non-selective for the various members of the PARP family and there is a need for selective inhibitors. In addition, new methods for the selectivity profiling of PARP inhibitors are needed.

One prerequisite for the identification and characterization of PARP inhibitors is the provision of suitable assays, preferably using physiological forms of the protein target. In the art, several strategies have been proposed to address this issue.

A two-step affinity purification protocol was described for the purification of PARP proteins from calf thymus (D'Amours et al., 1997. Analytical Biochemistry 249, 106-108). The first step consisted of DNA-cellulose chromatography and the second step of 3-Aminobenzamide affinity chromatography. 3-Aminobenzamide is a PARP inhibitor which interacts with the C-terminal catalytic domain of the enzyme.

Conventionally, PARP enzyme activity can be measured using purified or recombinant enzyme in a solution-based assay with a suitable substrate (Dantzer et al., 2006. Methods in Enzymology 409, 493-510). This type of assay can be used to identify PARP inhibitors, but also to assess inhibitor selectivity by testing an inhibitor against all members of the PARP family.

In view of the above, there is a need for providing effective tools and methods for the identification and selectivity profiling of PARP interacting compounds as well as for the purification of PARP proteins.

The present invention relates inter alia to an immobilization compound of formula (I)

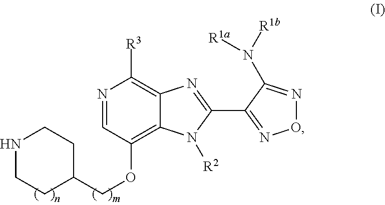

or a salt thereof, wherein
R^{1a}, R^{1b}, R² are independently selected from the group consisting of H; and C₁₋₄ alkyl, wherein C₁₋₄ alkyl is optionally substituted with one or more halogen, which are the same or different;
R³ is H; halogen; CN; C(O)OR⁴; OR⁴; C(O)R⁴; C(O)N(R⁴R^{4a}); S(O)₂N(R⁴R^{4a}); S(O)N(R⁴R^{4a}); S(O)₂R⁴; S(O)R⁴; SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R⁵, which are the same or different;
R⁴, R^{4a}, R^{4b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁵, which are the same or different;
R⁵ is halogen; CN; OR⁶; SR⁶; N(R⁶R^{6a}); or NO₂;
R⁶, R^{6a} are independently selected from the group consisting of H; and C₁₋₄ alkyl, wherein C₁₋₄ alkyl is optionally substituted with one or more halogen, which are the same or different;
m is 0; 1; or 2;
n is 0; 1; or 2.

In case a variable or substituent can be selected from a group of different variants and such variable or substituent occurs more than once the respective variants can be the same or different.

Within the meaning of the present invention the terms are used as follows:

“Alkyl” means a straight-chain or branched saturated hydrocarbon chain. Each hydrogen of an alkyl carbon may be replaced by a substituent.

“Alkenyl” means a straight-chain or branched hydrocarbon chain that contains at least one carbon-carbon double bond. Each hydrogen of an alkenyl carbon may be replaced by a substituent.

“Alkynyl” means a straight-chain or branched hydrocarbon chain, that contains at least one carbon-carbon triple bond. Each hydrogen of an alkynyl carbon may be replaced by a substituent.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —C(CH₂)—, —CH(C₂H₅)—, —CH(CH₃)₂—, when two moieties of a molecule are linked by the alkyl group. Each hydrogen of a C₁₋₄ alkyl carbon may be replaced by a substituent.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —C(CH₂)—, —CH₂—CH₂—CH₂—, —CH(C₂H₅)—, —CH(CH₃)₂—, when two moieties of a molecule are linked by the alkyl group. Each hydrogen of a C₁₋₆ alkyl carbon may be replaced by a substituent.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent.

“C₂₋₆ alkynyl” means an alkynyl chain having 2 to 6 carbon atoms, e.g. if present at the end of a molecule: —CH≡CH, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₆ alkynyl carbon may be replaced by a substituent.

“Halogen” means fluoro, chloro, bromo or iodo. It is generally preferred that halogen is fluoro or chloro.

The immobilization compounds claimed in the present invention have been named as “immobilization compounds” due to their preferred use in the preparation of immobilization products as described below. However, other possible uses, e.g. as a soluble competitor in assays or as a labelled probe, are also explicitly included within the present invention.

In case the immobilization compounds according to formula (I) contain one or more acidic or basic groups, the invention also comprises their corresponding salts. Thus, the immobilization compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Immobilization compounds of the formula (I) which contain one or more basic groups, i.e. groups which can be protonated, can be present and can be used according to the invention in the form of to their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the immobilization compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts.

The present invention furthermore includes all solvates of the immobilization compounds according to the invention.

As it can be taken from the Examples, immobilization compounds falling under formula (I) have been shown to bind to PARP proteins, which makes them useful tools in the context of assays for the identification of PARP interacting compounds.

Preferred immobilization compounds of formula (I) are those immobilization compounds in which one or more of the residues contained therein have the meanings given below, with all combinations of preferred substituent definitions being a subject of the present invention. With respect to all preferred immobilization compounds of the formulae (I) the present invention also includes all tautomeric and stereoisomeric forms and mixtures thereof in all ratios.

In preferred embodiments of the present invention, the substituents mentioned below independently have the following meaning. Hence, one or more of these substituents can have the preferred or more preferred meanings given below.

Preferably, R^{1a}, R^{1b} are independently selected from the group consisting of H; CH₃; and C₂H₅. In a more preferred embodiment at least one of R^{1a}, R^{1b} is H. In a further more preferred embodiment R^{1a}, R^{1b} are H.

Preferably, R² is H; CH₃; or C₂H₅. In a more preferred embodiment R² is CH₃ or C₂H₅, especially C₂H₅.

Preferably, R³ is halogen, especially chloro; or C₁₋₆ alkynyl, optionally substituted with R⁵.

Preferably R⁵ is OR⁶.

Preferably R⁶ is H; or CH₃.

Even more preferred is R³ chloro; or C≡C—C(CH₃)₂OH.

Preferably, m is 1. Preferably, n is 1. Preferably, n+m is 2.

Preferred immobilization compounds of formula (I) of the present invention are selected from the group consisting of

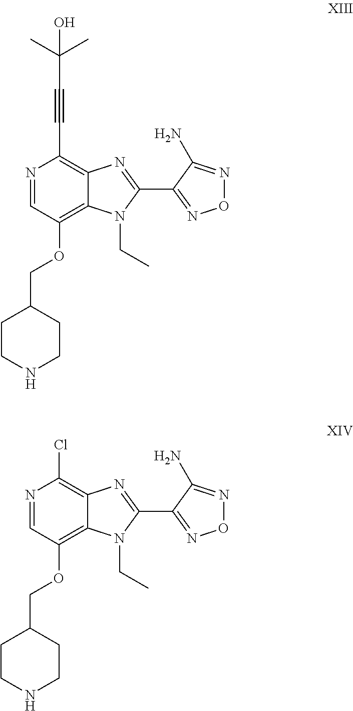

or a mixture of both, preferably in the form of a hydrohalogenide or carboxylate, especially as hydrochloride or formate.

The immobilization compounds of the present invention can be prepared by methods well known in the art. Exemplary analogous routes for the synthesis are described in FIG. 1.

The invention further relates to a method for the preparation of an immobilization product, wherein at least one immobilization compound according to the invention is immobilized on a solid support. Such immobilization products obtainable according to the method of the invention are e.g. useful in the methods of the invention for the identification of PARP interacting compounds or in diagnostic methods for the diagnosis of cancer.

According to the method of the invention, at least one immobilization compound of the invention is immobilized on a solid support. Throughout the invention, the term “solid support” relates to every undissolved support being able to immobilize a small molecule ligand on its surface.

According to the invention, the term “at least one immobilization compound” means either that at least one immobilization compound of the same type is immobilized on the solid support or that one or more different immobilization compounds (each of them either in singular or plural) may be immobilized on the solid support. Preferably, one or two different immobilization compounds are immobilized on the solid support, more preferably the preferred immobilization compounds of formula (I) of the present invention selected from the group consisting of

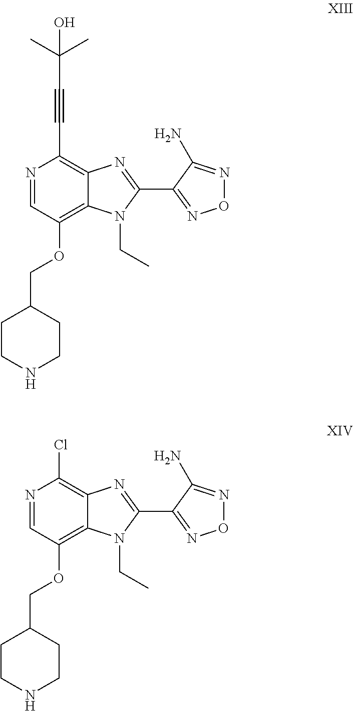

The solid support may be selected from the group consisting of agarose, modified agarose, sepharose beads (e.g. NHS-activated sepharose), latex, cellulose, and ferro- or ferrimagnetic particles.

In case that the solid support is a material comprising various entities, e.g. in case that the solid support comprises several beads or particles, it is envisaged within the present invention that, if different immobilization compounds are immobilized, on each single entity, e.g. each bead or particle, one or more different immobilization compounds are immobilized. Therefore, in case that two immobilization compounds are used, it is envisaged within the present invention that on each single entity one or two different immobilization compounds are immobilized. If no measures are taken that on one entity only one different immobilization compound is immobilized, it is very likely that on each entity all different immobilization compounds will be present.

The immobilization compound or compounds of the invention may be coupled to the solid support either covalently or non-covalently. Non-covalent binding includes binding via biotin affinity ligands binding to steptavidin matrices.

Preferably, the immobilization compound or compounds are covalently coupled to the solid support.

Methods for immobilizing compounds on solid supports are known in the art and further exemplified in Example 1.

In general, before the coupling, the matrixes can contain active groups such as NHS, Carbodimide etc. to enable the coupling reaction with the immobilization compound. The immobilization compound can be coupled to the solid support by direct coupling (e.g. using functional groups such as amino-, sulfhydryl-, carboxyl-, hydroxyl-, aldehyde-, and ketone groups) and by indirect coupling (e.g. via biotin, biotin being covalently attached to the immobilization product of the invention and non-covalent binding of biotin to streptavidin which is bound directly to the solid support).

The linkage to the solid support material may involve cleavable and non-cleavable linkers. The cleavage may be achieved by enzymatic cleavage or treatment with suitable chemical methods.

Therefore, according to a preferred embodiment of the invention, the immobilization product results from a covalent direct or linker mediated attachment of the at least one immobilization compound of the invention to the solid support.

The linker may be a C₁₋₁₀ alkylene group, which is optionally interrupted by one or more atoms or functional groups selected from the group consisting of S, O, NH, C(O)O, C(O), and C(O)NH and wherein the linker is optionally substituted with one or more substituents independently selected from the group consisting of halogen, OH, NH₂, C(O)H, C(O)NH₂, SO₃H, NO₂, and CN.

The term “C₁₋₁₀ alkylene” means an alkylene chain having 1-10 carbon atoms, e.g. methylene, ethylene, —CH═CH—, n-propylene and the like, wherein each hydrogen of a carbon atom may be replaced by a substituent.

The term “interrupted” means that the one or more atoms or functional groups are inserted between two carbon atoms of the alkylene chain or at the end of said chain.

Preferably, said immobilization occurs via the ring nitrogen atom of the saturated ring in formula (I) above. More preferred, said nitrogen atom is part of an amine functional group, so that the immobilization occurs via amid bond forming of an immobilization compound of the present invention or a mixture thereof and optionally activated carboxylic acid functional groups of the solid support. Perhaps well known protective group techniques may be required during the immobilization step.

The invention further relates to an immobilization product, obtainable by the method of the invention.

Therefore, an immobilization product which is obtainable by the method of the invention is an immobilization compound immobilized on a solid support. This immobilization product will be referred to in the following as the immobilization product of the invention and is used in the methods of the present invention.

In a preferred embodiment, the immobilization compound or immobilization product of the invention may further be labeled.

By “labeled” is meant that the respective substance is either directly or indirectly labeled with a molecule which provides a detection signal, e.g. radioisotope, fluorescent tag, chemiluminescent tag, a peptide or specific binding molecules. Specific binding molecules include pairs, such as biotin and streptavidin, digoxin and antidigoxin. The label can directly or indirectly provide a detectable signal. The tag can also be a peptide which can be used in an enzyme fragment complementation assay (e.g. beta-galactosidase enzyme fragment complementation; Zaman et al., 2006. Assay Drug Dev. Technol. 4(4):411-420). The labeled compounds would be useful not only in imaging techniques but also in assays, both in vitro and in vivo, for identifying PARP interacting compounds by inhibition of binding of the labeled compound, for example in PARP assays that contain such labeled compounds.

Radioisotopes are commonly used in biological applications for the detection of a variety of biomolecules and have proven to be useful in binding assays. Several examples of probes have been designed to incorporate ³H (also written as T for tritium) because it can replace hydrogen in a probe without altering its structure. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written D for Deuterium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I.

Guidance for the selection and methods for the attachment of fluorescent tags (e.g. fluorescein, rhodamine, dansyl, NBD (nitrobenz-2-oxa-1,3-diazole), BODIPY (dipyrromethene boron difluoride), and cyanine (Cy)-dyes) to small molecule ligands are generally known in the art. The application of fluorescent probes (fluorophores) in assays for high throughput screening (HTS) of enzymes was described (Zaman et al., 2003. Comb. Chem. High Throughput Screen 6(4): 313-320). The change of the fluorescent properties after binding of the fluorescent probe to the target protein can be determined by measuring for example fluorescence polarization, fluorescence resonance energy transfer, or fluorescence lifetime. In addition, the ALPHAScreen technology can be used where the excitation of a donor bead at 680 nm produces singlet oxygen which can diffuse to an acceptor bead undergoing a chemiluminescent reaction (Glickman et al., 2002. J. Biomol. Screen. 7(1):3-10).

As already discussed above, one possible use of the immobilization products of the invention is in the context of the identification of PARP. Therefore, the present invention also relates to such methods and uses.

In a first aspect of the methods of the invention, the invention therefore relates to a method for the identification of a PARP interacting compound, comprising the steps of
a) providing a protein preparation containing PARP,
b) contacting the protein preparation with the immobilization product of the invention under conditions allowing the formation of a complex between PARP and the immobilization product,
c) incubating the complex with a given compound, and
d) determining whether the compound is able to separate PARP from the immobilization product.

In a second aspect of the methods of the invention, the present invention relates to a method for the identification of a PARP interacting compound, comprising the steps of
a) providing a protein preparation containing PARP,
b) contacting the protein preparation with the immobilization product of the invention and with a given compound under conditions allowing the formation of a complex between PARP and the immobilization product, and
c) detecting the complex formed in step b).

In a third aspect of the methods of the invention, the invention provides a method for the identification of a PARP interacting compound, comprising the steps of:
a) providing two aliquots of a protein preparation containing PARP,
b) contacting one aliquot with the immobilization product of the invention under conditions allowing the formation of a complex between PARP and the immobilization product,
c) contacting the other aliquot with the immobilization product and with a given compound under conditions allowing the formation of the complex, and
d) determining the amount of the complex formed in steps b) and c).

In a fourth aspect of the methods of the invention, the invention relates to a method for the identification of a PARP interacting compound, comprising the steps of:
a) providing two aliquots comprising each at least one cell containing PARP,
b) incubating one aliquot with a given compound,
c) harvesting the cells of each aliquot,
d) lysing the cells in order to obtain protein preparations,
e) contacting the protein preparations with the immobilization product of the invention under conditions allowing the formation of a complex between PARP and the immobilization product, and
f) determining the amount of the complex formed in each aliquot in step e).

According to the present invention, the term “PARP” denotes one or more, especially all members of the PARP family (e.g. PARP1, PARP2, PARP3, PARP4 (VPARP), PARP5a (tankyrasel), PARPSb (tankyrase 2), PARP5c (tankyrase3), PARP6, PARP7 (TiPARP), PARP8, PARP9 (Bal), PARP10, PARP11, PARD12, PARP13, PARP14, PARP15, PARP16) (Ame et al., 2004. Bioessays 26(8):882-93). However, throughout the invention, it is preferred that PARP means PARP1, PARP10, PARP14, PARP15 or PARP16, especially the human isoforms thereof. Throughout the invention, the term “isoform” also includes PARP family members.

According to the present invention, the term “PARP” relates to both human and other proteins of this family. The expression especially includes functionally active derivatives thereof, or functionally active fragments thereof, or a homologues thereof, or variants encoded by a nucleic acid that hybridizes to the nucleic acid encoding said protein under low stringency conditions. Preferably, these low stringency conditions include hybridization in a buffer comprising 35% formamide, 5×SSC, 50 mM Tris-HCl (pH 7.5), 5 mM EDTA, 0.02% PVP, 0.02% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1-5 hours at 55° C., and washing in a buffer consisting of 2×SSC, 25 mM Tris-HCl (pH 7.4) 5 mM EDTA, and 0.1% SDS for 1.5 hours at 60° C.

The compounds identified in the context of the presence invention may be a ligand for one, some or all isoforms of PARP (see above).

In some aspects of the invention, first a protein preparation containing PARP is provided. The methods of the present invention can be performed with any protein preparation as a starting material, as long as the PARP is solubilized in the preparation. Examples include a liquid mixture of several proteins, a cell lysate, a partial cell lysate which contains not all proteins present in the original cell or a combination of several cell lysates. The term “protein preparation” also includes dissolved purified protein.

The presence of PARP protein species in a protein preparation of interest can be detected on Western blots probed with antibodies that are specifically directed against PARP. In case that PARP is a specific isoform (e.g. PARP1), the presence of said isoform can be determined by an isoform-specific antibody. Such antibodies are known in the art (Cheong et al., 2003. Clin. Cancer. Res. 9(13):5018-27; Ame et al., 1999. J. Biol. Chem. 274(25):17860-17868). Alternatively, also mass spectrometry (MS) could be used to detect PARP, especially isoforms of PARP (see below).

Cell lysates or partial cell lysates can be obtained by isolating cell organelles (e.g. nucleus, mitochondria, ribosomes, golgi etc.) first and then preparing protein preparations derived from these organelles. Methods for the isolation of cell organelles are known in the art. In addition, methods for the preparation of cell lysates have been described (Dantzer et al., 2006. Methods in Enzymology 409, 493-510).

In addition, protein preparations can be prepared by fractionation of cell extracts thereby enriching specific types of proteins such as cytoplasmic or membrane proteins.

Furthermore protein preparations from body fluids can be used (e.g. blood, cerebrospinal fluid, peritoneal fluid and urine).

For example whole embryo lysates derived from defined development stages or adult stages of model organisms such as C. elegans can be used. In addition, whole organs such as heart dissected from mice can be the source of protein preparations. These organs can also be perfused in vitro in order to obtain a protein preparation.

Furthermore, the protein preparation may be a preparation containing PARP which has been recombinantely produced. Methods for the production of recombinant proteins in prokaryotic and eukaryotic cells are widely established (Ame et al., 1999. J. Biol. Chem. 274(25):17860-17868).

In a preferred embodiment of the methods of the invention, the provision of a protein preparation includes the steps of harvesting at least one cell containing PARP and lysing the cell.

Suitable cells for this purpose are e.g. those cells or tissues were members of the PARP family are expressed.

Therefore, in a preferred embodiment, cells isolated from peripheral blood represent a suitable biological material. Procedures for the preparation and culture of human lymphocytes and lymphocyte subpopulations obtained from peripheral blood (PBLs) are widely known (W. E. Biddison, Chapter 2.2 “Preparation and culture of human lymphocytes” in Current Protocols in Cell Biology, 1998, John Wiley & Sons, Inc.). For example, density gradient centrifugation is a method for the separation of lymphocytes from other blood cell populations (e.g. erythrocytes and granulocytes). Human lymphocyte subpopulations can be isolated via their specific cell surface receptors which can be recognized by monoclonal antibodies. The physical separation method involves coupling of these antibody reagents to magnetic beads which allow the enrichment of cells that are bound by these antibodies (positive selection). The isolated lymphocyte cells can be further cultured and stimulated by adding cytokines to initiate receptor-mediated cell signaling and subsequently phosphorylation of STAT proteins (Schindler et al., 2007. 282(28):20059-20063).

As an alternative to primary human cells cultured cell lines (e.g. MOLT-4 cells, Jurkat or Ramos cells) can be used.

In a preferred embodiment, the cell is part of a cell culture system and methods for the harvest of a cell out of a cell culture system are known in the art (literature supra).

The choice of the cell will mainly depend on the expression of PARP, since it has to be ensured that the protein is principally present in the cell of choice. In order to determine whether a given cell is a suitable starting system for the methods of the invention, methods like Westernblot, PCR-based nucleic acids detection methods, Northernblots and DNA-microarray methods (“DNA chips”) might be suitable in order to determine whether a given protein of interest is present in the cell.

The choice of the cell may also be influenced by the purpose of the study. If the in vivo efficacy for a given drug needs to be analyzed then cells or tissues may be selected in which the desired therapeutic effect occurs (e.g. B-cells). By contrast, for the elucidation of protein targets mediating unwanted side effects the cell or tissue may be analysed in which the side effect is observed (e.g. cardiomyocytes, vascular smooth muscle or epithelium cells).

Furthermore, it is envisaged within the present invention that the cell containing PARP may be obtained from an organism, e.g. by biopsy. Corresponding methods are known in the art. For example, a biopsy is a diagnostic procedure used to obtain a small amount of tissue, which can then be examined miscroscopically or with biochemical methods. Biopsies are important to diagnose, classify and stage a disease, but also to evaluate and monitor drug treatment.

It is encompassed within the present invention that by the harvest of the at least one cell, the lysis is performed simultaneously. However, it is equally preferred that the cell is first harvested and then separately lysed.

Methods for the lysis of cells are known in the art. Lysis of different cell types and tissues can be achieved by homogenizers (e.g. Potter-homogenizer), ultrasonic desintegrators, enzymatic lysis, detergents (e.g. NP-40, Triton X-100, CHAPS, SDS), osmotic shock, repeated freezing and thawing, or a combination of these methods.

According to the methods of the invention, the protein preparation containing PARP is contacted with the immobilization product under conditions allowing the formation of a complex between PARP and the immobilization product of the invention.

In the present invention, the term “a complex between PARP and the immobilization product” denotes a complex where the immobilization product interacts with PARP, e.g. by covalent or, most preferred, by non-covalent binding.

The skilled person will know which conditions can be applied in order to enable the formation of said complex.

In the context of the present invention, the term “under conditions allowing the formation of the complex” includes all conditions under which such formation, preferably such binding is possible. This includes the possibility of having the solid support on an immobilized phase and pouring the lysate onto it. In another preferred embodiment, it is also included that the solid support is in a particulate form and mixed with the cell lysate.

In the context of non-covalent binding, the binding between the immobilization product and PARP is, e.g., via salt bridges, hydrogen bonds, hydrophobic interactions or a combination thereof.

In a preferred embodiment, the steps of the formation of said complex are performed under essentially physiological conditions. The physical state of proteins within cells is described in Petty, 1998 (Howard R. Petty, Chapter 1, Unit 1.5 in: Juan S. Bonifacino, Mary Dasso, Joe B. Harford, Jennifer Lippincott-Schwartz, and Kenneth M. Yamada (eds.) Current Protocols in Cell Biology Copyright© 2003 John Wiley & Sons, Inc. All rights reserved. DOI: 10.1002/0471143030.cb0101s00Online Posting Date: May, 2001Print Publication Date: October, 1998).

The contacting under essentially physiological conditions has the advantage that the interactions between the immobilization product, the cell preparation containing PARP and optionally the compound reflect as much as possible the natural conditions. “Essentially physiological conditions” are inter alia those conditions which are present in the original, unprocessed sample material. They include the physiological protein concentration, pH, salt concentration, buffer capacity and post-translational modifications of the proteins involved. The term “essentially physiological conditions” does not require conditions identical to those in the original living organism, wherefrom the sample is derived, but essentially cell-like conditions or conditions close to cellular conditions. The person skilled in the art will, of course, realize that certain constraints may arise due to the experimental set-up which will eventually lead to less cell-like conditions. For example, the eventually necessary disruption of cell walls or cell membranes when taking and processing a sample from a living organism may require conditions which are not identical to the physiological conditions found in the organism. Suitable variations of physiological conditions for practicing the methods of the invention will be apparent to those skilled in the art and are encompassed by the term “essentially physiological conditions” as used herein. In summary, it is to be understood that the term “essentially physiological conditions” relates to conditions close to physiological conditions, as e.g. found in natural cells, but does not necessarily require that these conditions are identical.

For example, “essentially physiological conditions” may comprise 50-200 mM NaCl or KCl, pH 6.5-8.5, 20-37° C., and 0.001-10 mM divalent cation (e.g. Mg++, Ca++); more preferably about 150 m NaCl or KCl, pH7.2 to 7.6, 5 mM divalent cation and often include 0.01-1.0 percent non-specific protein (e.g. BSA). A non-ionic detergent (Tween, NP-40, Triton-X100) can often be present, usually at about 0.001 to 2%, typically 0.05-0.2% (volume/volume). For general guidance, the following buffered aequous conditions may be applicable: 10-250 mM NaCl, 5-50 mM Tris HCl, pH5-8, with optional addition of divalent cation(s) and/or metal chelators and/or non-ionic detergents.

Preferably, “essentially physiological conditions” mean a pH of from 6.5 to 7.5, preferably from 7.0 to 7.5, and/or a buffer concentration of from 10 to 50 mM, preferably from 25 to 50 mM, and/or a concentration of monovalent salts (e.g. Na or K) of from 120 to 170 mM, preferably 150 mM. Divalent salts (e.g. Mg or Ca) may further be present at a concentration of from 1 to 5 mM, preferably 1 to 2 mM, wherein more preferably the buffer is selected from the group consisting of Tris-HCl or HEPES.

The skilled person will appreciate that between the individual steps of the methods of the invention, washing steps may be necessary. Such washing is part of the knowledge of the person skilled in the art. The washing serves to remove non-bound components of the cell lysate from the solid support. Nonspecific (e.g. simple ionic) binding interactions can be minimized by adding low levels of detergent or by moderate adjustments to salt concentrations in the wash buffer.

According to the identification methods of the invention, the read-out system is either the detection or determination of PARP (first aspect of the invention), the detection of the complex between PARP and the immobilization product (second aspect of the invention), or the determination of the amount of the complex between PARP and the immobilization product (second, third and fourth aspect of the invention).

In the method according to the first aspect of the invention, the detection or determination of the amount of separated PARP is preferably indicative for the fact that the compound is able to separate PARP from the immobilization product. This capacity indicates that the respective compound interacts, preferably binds to PARP, which is indicative for its therapeutic potential.

In one embodiment of the method according to the second aspect of the invention, the complex formed during the method of the invention is detected. The fact that such complex is formed preferably indicates that the compound does not completely inhibit the formation of the complex. On the other hand, if no complex is formed, the compound is presumably a strong interactor with PARP, which is indicative for its therapeutic potential.

According to the methods of the second, third and fourth aspect of the invention the amount of the complex formed during the method is determined. In general, the less complex in the presence of the respective compound is formed, the stronger the respective compound interacts with PARP, which is indicative for its therapeutic potential.

The detection of the complex formed according to the second aspect of the invention can be performed by using labeled antibodies directed against PARP and a suitable readout system.

According to a preferred embodiment of the second aspect of the invention, the complex between PARP and the immobilization product is detected by determining its amount.

In the course of the second, third and fourth aspect of the invention, it is preferred that PARP is separated from the immobilization product in order to determine the amount of said complex.

According to invention, separating means every action which destroys the interactions between the immobilization compound and PARP. This includes in a preferred embodiment the elution of PARP from the immobilization compound.

The elution can be achieved by using non-specific reagents as described in detail below (ionic strength, pH value, detergents). In addition, it can be tested whether a compound of interest can specifically elute the PARP from the immobilization compound. Such PARP interacting compounds are described further in the following sections.

Such non-specific methods for destroying the interaction are principally known in the art and depend on the nature of the ligand enzyme interaction. Principally, change of ionic strength, the pH value, the temperature or incubation with detergents are suitable methods to dissociate the target enzymes from the immobilized ligand. The application of an elution buffer can dissociate binding partners by extremes of pH value (high or low pH; e.g. lowering pH by using 0.1 M citrate, pH2-3), change of ionic strength (e.g. high salt concentration using NaI, KI, MgCl₂, or KCl), polarity reducing agents which disrupt hydrophobic interactions (e.g. dioxane or ethylene glycol), or denaturing agents (chaotropic salts or detergents such as Sodium-docedyl-sulfate, SDS).

In some cases, the solid support has preferably to be separated from the released material. The individual methods for this depend on the nature of the solid support and are known in the art. If the support material is contained within a column the released material can be collected as column flowthrough. In case the support material is mixed with the lysate components (so called batch procedure) an additional separation step such as gentle centrifugation may be necessary and the released material is collected as supernatant. Alternatively magnetic beads can be used as solid support so that the beads can be eliminated from the sample by using a magnetic device.

In step d) of the method according to the first aspect of the invention, it is determined if PARP has been separated from the immobilization product of the invention. This may include the detection of PARP or the determination of the amount PARP.

Consequently, at least in preferred embodiments of all identification methods of the invention, methods for the detection of separated PARP or for the determination of its amount are used. Such methods are known in the art and include physico-chemical methods such as protein sequencing (e.g. Edmann degradation), analysis by mass spectrometry methods or immunodetection methods employing antibodies directed against PARP.

Throughout the invention, if an antibody is used in order to detect PARP or in order to determine its amount (e.g. via ELISA), the skilled person will understand that, if a specific isoform of PARP is to be detected or if the amount of a specific isoform of PARP is to be determined, an isoform-specific antibody may be used. As indicated above, such antibodies are known in the art. Furthermore, the skilled person is aware of methods for producing the same.

For example, PARP2 can be specifically detected with an antibody directed at PARP2 that does not crossreact with other PARP family members (Ame et al., 1999. J. Biol. Chem. 274(25):17860-17868).

Alternatively, several isoforms of PARP can be detected by mass spectrometry methods as illustrated in Example 2.

Preferably, PARP is detected or the amount of PARP is determined by mass spectrometry or immunodetection methods.

The identification of proteins with mass spectrometric analysis (mass spectrometry) is known in the art (Shevchenko et al., 1996. Analytical Chemistry 68: 850-858) and is further illustrated in the example section.

Preferably, the mass spectrometry analysis is performed in a quantitative manner, for example by using iTRAQ technology (isobaric tags for relative and absolute quatification) or cICAT (cleavable isotope-coded affinity tags) (Wu et al., 2006. J. Proteome Res. 5, 651-658).

According to a further preferred embodiment of the present invention, the characterization by mass spectrometry (MS) is performed by the identification of proteotypic peptides of PARP. The idea is that PARP is digested with proteases and the resulting peptides are determined by MS. As a result, peptide frequencies for peptides from the same source protein differ by a great degree, the most frequently observed peptides that “typically” contribute to the identification of this protein being termed “proteotypic peptide”. Therefore, a proteotypic peptide as used in the present invention is an experimentally well observable peptide that uniquely identifies a specific protein or protein isoform.

According to a preferred embodiment, the characterization is performed by comparing the proteotypic peptides obtained in the course of practicing the methods of the invention with known proteotypic peptides. Since, when using fragments prepared by protease digestion for the identification of a protein in MS, usually the same proteotypic peptides are observed for a given enzyme, it is possible to compare the proteotypic peptides obtained for a given sample with the proteotypic peptides already known for enzymes of a given class of enzymes and thereby identifying the enzyme being present in the sample.

As an alternative to mass spectrometry analysis, the eluted PARP (including coeluted binding partners or scaffold proteins), can be detected or its amount can be determined by using a specific antibody directed against PARP (or against an isoform of PARP, see above).

Furthermore, in another preferred embodiment, once the identity of the coeluted binding partner has been established by mass spectrometry analysis, each binding partner can be detected with specific antibodies directed against this protein.

Suitable antibody-based assays include but are not limited to Western blots, ELISA assays, sandwich ELISA assays and antibody arrays or a combination thereof. The establishment of such assays is known in the art (Chapter 11, Immunology, pages 11-1 to 11-30 in: Short Protocols in Molecular Biology. Fourth Edition, Edited by F. M. Ausubel et al., Wiley, New York, 1999).

These assays can not only be configured in a way to detect and quantify a PARP interacting protein of interest (e.g. a catalytic or regulatory subunit of a PARP complex), but also to analyse posttranslational modification patterns such as phosphorylation, ubiquitin modification or poly(ADP)-ribosylation (Affar et al., 1999. Biochimica et Biophysica Acta 1428, 137-146).

Furthermore, the identification methods of the invention involve the use of compounds which are tested for their ability to be a PARP interacting compound.

Principally, according to the present invention, such a compound can be every molecule which is able to interact with PARP, e.g. by inhibiting its binding to the immobilization product of the invention. Preferably, the compound has an effect on PARP, e.g. a stimulatory or inhibitory effect.

Preferably, said compound is selected from the group consisting of synthetic or naturally occurring chemical compounds or organic synthetic drugs, more preferably small molecule organic drugs or natural small molecule compounds. Preferably, said compound is identified starting from a library containing such compounds. Then, in the course of the present invention, such a library is screened.

Such small molecules are preferably not proteins or nucleic acids. Preferably, small molecules exhibit a molecular weight of less than 1000 Da, more preferred less than 750 Da, most preferred less than 500 Da.

A “library” according to the present invention relates to a (mostly large) collection of (numerous) different chemical entities that are provided in a sorted manner that enables both a fast functional analysis (screening) of the different individual entities, and at the same time provide for a rapid identification of the individual entities that form the library. Examples are collections of tubes or wells or spots on surfaces that contain chemical compounds that can be added into reactions with one or more defined potentially interacting partners in a high-throughput fashion. After the identification of a desired “positive” interaction of both partners, the respective compound can be rapidly identified due to the library construction. Libraries of synthetic and natural origins can either be purchased or designed by the skilled artisan.

Examples of the construction of libraries are known in the art, wherein natural products are used that are biologically validated starting points for the design of combinatorial libraries, as they have a proven record of biological relevance. This special role of natural products in medicinal chemistry and chemical biology can be interpreted in the light of new insights about the domain architecture of proteins gained by structural biology and bioinformatics. In order to fullfill the specific requirements of the individual binding pocket within a domain family it may be necessary to optimise the natural product structure by chemical variation. Solid-phase chemistry is said to become an efficient tool for this optimisation process. Collections of small molecules with diverse structures and “drug-like” properties have, in the past, been acquired by several means: by archive of previous internal lead optimisation efforts, by purchase from compound vendors, and by union of separate collections following company mergers. Although high throughput/combinatorial chemistry is described as being an important component in the process of new lead generation, the selection of library designs for synthesis and the subsequent design of library members has evolved to a new level of challenge and importance. The potential benefits of screening multiple small molecule compound library designs against multiple biological targets offers substantial opportunity to discover new lead structures.

In a preferred embodiment of the second and third aspect of the invention, the PARP containing protein preparation is first incubated with the compound and then with the immobilization product. However, the simultaneous incubation of the compound and the immobilization product of the invention (coincubation) with the PARP containing protein preparation is equally preferred (competitive binding assay).

In case that the incubation with the compound is first, the PARP is preferably first incubated with the compound for 10 to 60 minutes, more preferred 30 to 45 minutes at a temperature of 4° C. to 37° C., more preferred 4° C. to 25° C., most preferred 4° C. Preferably compounds are used at concentrations ranging from 1 nM to 100 μM, preferably from 10 nM to 10 μM. The second step, contacting with the immobilized ligand, is preferably performed for 10 to 60 minutes at 4° C.

In case of simultaneous incubation, the PARP is preferably simultaneously incubated with the compound and the immobilization product of the invention for 30 to 120 minutes, more preferred 60 to 120 minutes at a temperature of 4° C. to 37° C., more preferred 4° C. to 25° C., most preferred 4° C. Preferably compounds are used at concentrations ranging from 1 nM to 100 μM, preferably from 10 nM to 10 μM.

Furthermore, steps a) to c) of the second aspect of the invention may be performed with several protein preparations in order to test different compounds. This embodiment is especially interesting in the context of medium or high throughput screenings (see below).

In a preferred embodiment of the method of the invention according to the third or fourth aspect, the amount of the complex formed in step c) is compared to the amount formed in step b)

In a preferred embodiment of the method of the invention according to the third or fourth aspect, a reduced amount of the complex formed in step c) in comparison to step b) indicates that PARP is a target of the compound. This results from the fact that in step c) of this method of the invention, the compound competes with the ligand for the binding of PARP. If less PARP is present in the aliquot incubated with the compound, this means preferably that the compound has competed with the inhibitor for the interaction with the enzyme and is, therefore, a direct target of the protein and vice versa.

Preferably, the identification methods of the invention are performed as a medium or high throughput screening.

The interaction compound identified according to the present invention may be further characterized by determining whether it has an effect on PARP, for example on its enzymatic activity (Dantzer et al., 2006. Methods in Enzymology 409, 493-510).

Briefly, PARP enzyme activity (auto poly-ADP-ribosylation) can e.g. be measured by incubating purified PARP in a suitable buffer together with DNaseI treated DNA and [³²P]-NAD at room temperature. The reaction is stopped by adding trichloro acetic acid (TCA) and then filtered through a glass microfibre filter whereby the reaction product polyADP-ribose is retained on the filter. The filter is washed, dried and the amount of radioactivity is measured with a liquid scintillatin counter (Dantzer et al., 2006. Methods in Enzymology 409, 493-510).

The compounds identified according to the present invention may further be optimized (lead optimisation). This subsequent optimisation of such compounds is often accelerated because of the structure-activity relationship (SAR) information encoded in these lead generation libraries. Lead optimisation is often facilitated due to the ready applicability of high-throughput chemistry (HTC) methods for follow-up synthesis.

The invention further relates to a method for the preparation of a pharmaceutical composition comprising the steps of
a) identifying a PARP interacting compound as described above, and
b) formulating the interacting compound to a pharmaceutical composition.

Therefore, the invention provides a method for the preparation of pharmaceutical compositions, which may be administered to a subject in an effective amount. In a preferred aspect, the therapeutic is substantially purified. The subject to be treated is preferably an animal including, but not limited to animals such as cows, pigs, horses, chickens, cats, dogs, etc., and is preferably a mammal, and most preferably human. In a specific embodiment, a non-human mammal is the subject.

The compounds identified according to the invention are useful for the prevention or treatment of diseases where PARP plays a role (for example PARPI inhibitors for cancer). Consequently, the present invention also relates to the use of a compound identified by the methods of the invention for the preparation of a medicament for the treatment of one or more of the above mentioned diseases. Furthermore, the present invention relates to a pharmaceutical composition comprising said compound.

In general, the pharmaceutical compositions of the present invention comprise a therapeutically effective amount of a therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. In general, suppositories may contain active ingredient in the range of 0.5% to 10% by weight; oral formulations preferably contain 10% to 95% active ingredient.

Various delivery systems are known and can be used to administer a therapeutic of the invention, e.g., encapsulation in liposomes, microparticles, and microcapsules: use of recombinant cells capable of expressing the therapeutic, use of receptor-mediated endocytosis; construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, to attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

The invention further relates to a method for the purification of PARP, comprising the steps of
a) providing a protein preparation containing PARP,
b) contacting the protein preparation with the immobilization product of the invention under conditions allowing the formation of a complex between PARP and the immobilization product, and
c) separating PARP from the immobilization product.

As mentioned above, it has been surprisingly found that the compound of the invention and therefore also the immobilization product of the invention is a ligand which recognizes PARP proteins. This enables efficient purification methods for PARP proteins.

With respect to PARP, the protein preparation containing PARP, the conditions for contacting with the immobilization product of the invention, the immobilization product of the invention, the complex between PARP and the immobilization product of the invention, the separation of PARP from the immobilization product of the invention, and the detection of PARP or the determination of its amount, the embodiments as defined above for the identification methods of the invention also apply to the purification method of the invention.

In a preferred embodiment, the method of purification further comprises the step of purifying a specific isoform of PARP.

Preferably, said purification is performed using an isoform specific antibody as explained above.

In a preferred embodiment, the purification method of the invention further comprises after step c) the identification of proteins being capable of binding to PARP. This is especially interesting when the formation of a protein complex is performed under essentially physiological conditions, because it is then possible to preserve the natural condition of the enzyme which includes the existence of binding partners, enzyme subunits or post-translational modifications, which can then be identified with the help of mass spectrometry (MS).

Consequently, in a preferred embodiment, the purification method of the invention further comprises after step c) the determination whether the PARP is further posttranslationally modified, e.g. by ubiquitin modification or by poly(ADP)-ribosylation.

The binding proteins or the posttranslational modifications can be determined as explained above for the detection of PARP or the determination of the amount of PARP. Preferably, said methods include mass spectrometry of immunodetection methods as described above.

The invention further relates to a method for determining the presence of PARP in a sample, comprising the steps of:
a) providing a protein preparation expected to contain PARP,
b) contacting the protein preparation with the immobilization product of the invention under conditions allowing the formation of a complex between PARP and the immobilization product, and
c) detecting whether PARP has formed a complex with the immobilization product.

In a preferred embodiment of the invention, said detecting in step c) is performed by separating PARP from the immobilization product and further identification of PARP.

Said identification may be performed by mass spectrometry or immunodetection methods as described above.

With respect to PARP, the protein preparation containing PARP, the conditions for contacting with the immobilization product of the invention, the immobilization product of the invention, the complex between PARP and the immobilization product of the invention, the separation of PARP from the immobilization product of the invention, and the detection of PARP or the determination of its amount, the embodiments as defined above for the identification methods of the invention also apply to the purification method of the invention.

The invention further relates to the use of compound or the immobilization product of the invention for the identification of PARP interacting compounds and for the purification of PARP proteins. The embodiments as defined above also apply to the uses of the invention.

The invention further relates to a kit comprising the compound or the immobilization product of the invention. Such a kit is especially useful for performing the methods of the invention. Further components of the kit may be antibodies for the detection of PARP proteins, for example antibodies specific for PARP1 and antibodies directed at phosphorylation sites of PARP proteins. Such antibodies and their use are known in the art and they are commercially available (Cheong et al., 2003. Clin. Cancer. Res. 9(13):5018-27; Ame et al., 1999. J. Biol. Chem. 274(25):17860-17868). In addition, the kit may contain antibodies directed at poly-ADP-ribose (Affar et al., 1999. Biochimica et Biophysica Acta 1428, 137-146; Kawamitsu et al., 1984. Biochemistry 23(16):3771-3777). Furthermore, the kit may contain further auxiliary components like buffers, means for the detection of antibodies, positive controls, etc. Such components are known in the art.

The invention is further illustrated by the following figures and examples, which are not considered as being limiting for the scope of protection conferred by the claims of the present application. In case where in the following examples the term “affinity matrix” is used, this term refers to an immobilization product as defined in the present application.

### SHORT DESCRIPTION OF THE FIGURES

FIG. 1: Reaction scheme for the synthesis of 4-(2-(4-amino-1,2,5-oxadiazol-3-yl)-1-ethyl-7-(piperidin-4-ylmethoxy)-1H-imidazo[4,5-c]pyridin-4-yl)-2-methylbut-3-yn-2-ol (XIII) and 4-(4-chloro-1-ethyl-7-(piperidin-4-ylmethoxy)-1H-imidazo[4,5-c]pyridin-2-yl)-1,2,5-oxadiazol-3-amine (XIV). The compounds were synthesized as described in example 1. Step a) Br₂, H₂O, rt then 50° C. b) POCl₃, N,N-diethylaniline, 0° C. then reflux c) Ethylamine, rt d) HCl conc, 5 nCl₂, reflux e) Cyanoacetic acid, EDC, DCM, N-methylmorpholine, rt f) Acetic acid, 100° C. g) NaNO₂, rt h) Hydroxylamine, Et₃N, Dioxane, reflux i) isopropylmagnesium chloride, B(OMe)₃, H₂O₂, −78° C. j) tert-butyl 4-(bromomethyl)piperidine-1-carboxylate, CsCO₃, DMF, 40° C. k) 2-methyl-3-butyn-2-ol, tetrakis triphenylphosphine palladium (0), Zn, NaI, DBU, DMSO, 80° C. l) 4N HCl in Dioxane, MeOH.

FIG. 2: Structure of 4-(2-(4-amino-1,2,5-oxadiazol-3-yl)-1-ethyl-7-(piperidin-4-ylmethoxy)-1H-imidazo[4,5-c]pyridin-4-yl)-2-methylbut-3-yn-2-ol (XIII).

FIG. 3: Structure of 4-(4-chloro-1-ethyl-7-(piperidin-4-ylmethoxy)-1H-imidazo[4,5-c]pyridin-2-yl)-1,2,5-oxadiazol-3-amine (XIV).

FIG. 4: Drug pulldown experiment with the immobilization compound XIII.

A protein gel after staining with Coomassie brilliant blue is shown. The drug pulldown experiment was performed as described in example 2 with a 1:1 mixture of Jurkat and Ramos cell lysates containing 50 mg of protein. Proteins bound to the affinity matrix were eluted with SDS sample buffer and separated by SDS-polyacrylamide gel electrophoresis. The indicated gel areas were cut out as gel slices, proteins were treated with trypsin and subjected to analysis by mass spectrometry.

FIG. 5: Drug pulldown experiment with the immobilization compound XIV.

A protein gel after staining with Coomassie brilliant blue is shown. The drug pulldown experiment was performed as described in example 2 with a 1:1 mixture of Jurkat and Ramos cell lysates containing 50 mg of protein. Proteins bound to the affinity matrix were eluted with SDS sample buffer and separated by SDS-polyacrylamide gel electrophoresis. The indicated gel areas were cut out as gel slices, proteins were treated with trypsin and subjected to analysis by mass spectrometry.

FIG. 6: Amino acid sequence of human PARP1 (IP100449049.5). Peptides identified by mass spectrometry after a drug pulldown experiment with immobilization compound XIII are shown in bold type and underlined.

FIG. 7: Amino acid sequence of human PARP10 (IP100064457.3). Peptides identified by mass spectrometry after a drug pulldown experiment with immobilization compound XIII are shown in bold type and underlined.

FIG. 8: Amino acid sequence of human PARP 14 (IP100291215.5). Peptides identified by mass spectrometry after a drug pulldown with immobilization compound XIII are in bold type and underlined.

FIG. 9: Amino acid sequence of human PARP15 (IP100166182.3). Peptides identified by mass spectrometry after a drug pulldown with immobilization compound XIII are in bold type and underlined.

FIG. 10: Amino acid sequence of human PARP16 (IP100297151.3). Peptides identified by mass spectrometry after a drug pulldown with immobilization compound XIII are in bold type and underlined.

### EXAMPLES

### Example 1

### Preparation of the Immobilization Compound and Immobilization Product (Affinity Matrix)

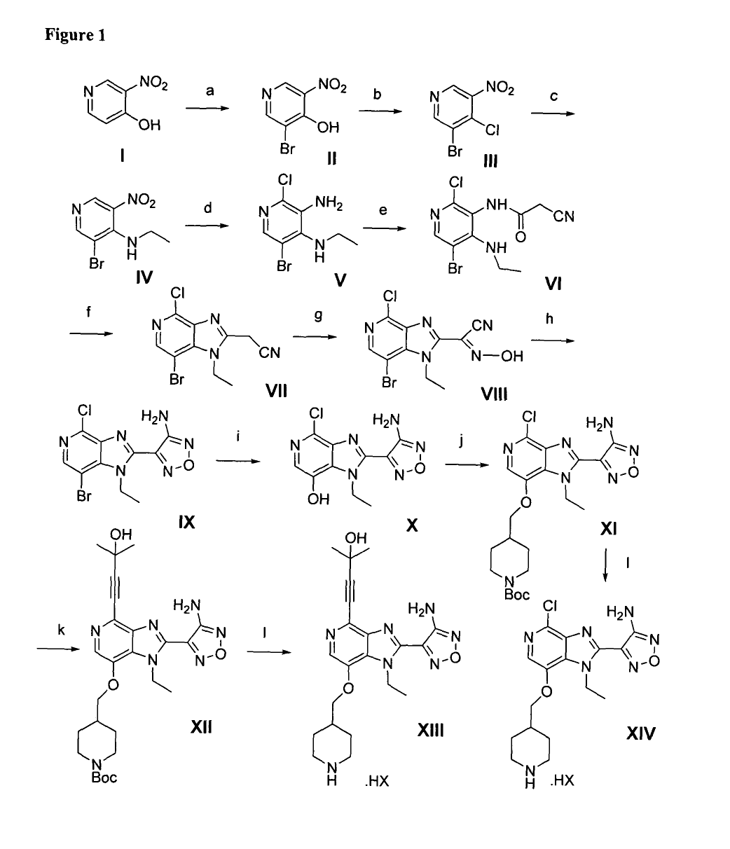

This example describes the synthesis of compounds and methods for their immobilization on a solid support yielding the immobilization product (affinity matrix) used in the following examples for the capturing of PARP proteins from cell lysates. The synthesis of compounds is illustrated in synthetic scheme 1 (FIG. 1).

### Synthesis of 3-bromo-5-nitropyridin-4-ol (II)

To a suspension of 4-hydroxy-3-nitropyridine (I) (7.0 g, 50 mmol) in water (50 ml) was added bromide (3.23 ml, 63 mmol) drop wise at room temperature. The resulting mixture was stirred for 1 hour then heated at 50° C. for 2 hours. After cooling to room temperature and stirring a further 1 hour, the product was filtered, washed with cold water and dried under vacuum for 3 days to yield the desired product as a white solid (8.35 g, 76%). LCMS Rt=0.45 nm, no significant MS trace.

### Synthesis of 3-bromo-4-chloro-5-nitropyridine (III)

To phosphorus oxychloride (50 ml) cooled in ice was slowly added 3-bromo-5-nitropyridin-4-ol (6.57 g, 30 mmol). The resulting suspension was stirred at 0° C. and N,N-diethylaniline (4.77 ml, 30 mmol) was added drop wise. The resulting mixture was warmed at room temperature, then refluxed for 2 hours. The resulting black solution was concentrated under vacuum and the residue poured onto ice. The mixture was extracted with ether (200 ml). The organic layer was washed with water twice, brine, and dried on MgSO₄. After filtration the solvent was removed to yield the desired compound as a brown oil which solidified upon further drying (6.46 g, 85%) LCMS Rt=3.18 nm, no significant MS trace.

### Synthesis of 3-bromo-N-ethyl-5-nitropyridin-4-amine (IV)

To a solution of 3-bromo-4-chloro-5-nitropyridine (6.45 g, 25.4 mmol) in THF (19 ml) was added slowly a solution of ethylamine in water (70% solution, 101 mmol, 13 ml). The solution was stirred at room temperature for 3 hours then poured into water. The resulting solution was extracted twice with ethyl acetate. The organic layer was washed with brine, then dried over MgSO4. The solvent was removed. The crude product was purified by flash chromatography (ethyl acetate-Hexane 1:9 to 3:7) to yield the desired compound as a brown oil (5.45 g, 92%) LCMS Rt=2.82 nm [M+H]⁺=246-248.

### Synthesis of 5-bromo-2-chloro-N4-ethylpyridine-3,4-diamine (V)

3-bromo-N-ethyl-5-nitropyridin-4-amine (4.68 g, 19 mmol) was dissolved in concentrated hydrochloric acid (47 ml) and heated at 85° C. Tin Chloride (10.8 g, 57 mmol) was added in portions. The reaction was heated at reflux for 1 hour then allowed to cool to room temperature overnight. The off white solid was filtered off then suspended in icy water (90 ml). The pH was adjusted to 12 by addition of 12N sodium hydroxide. The resulting solution was extracted with ethyl acetate (2×120 ml). The organic layer was washed with brine, and dried over MgSO4. The solvent was removed to yield the desired compound as a yellow oil (3.98 g, 83%) LCMS Rt=3.07 nm [M+H]⁺=249.9-251.9.

### Synthesis of N-(5-bromo-2-chloro-4-(ethylamino)pyridin-3-yl)-2-cyanoacetamide (VI)

To a solution of 5-bromo-2-chloro-N4-ethylpyridine-3,4-diamine (3.98 g, 15.9 mmol) in dichloromethane (40 ml) was added EDC (4.57 g, 23.8 mmol), cyanoacetic acid (2.02 g, 23.8 mmol) and N-methylmorpholine (6.98 ml, 63.5 mmol). The reaction was stirred at room temperature for 4 hours. The solvent was removed under vacuum. To the slurry was added 150 ml of warm ethyl acetate (40° C.). The organic layer was washed with 2×125 ml of warm water (40° C.), brine (200 ml) then dried over MgSO4. The solvent was partially removed to obtain a slurry which was filtered. The white solid was washed with 2×20 ml of cold water and dried in a vacuum oven at 40° C. to yield the desired product (3.66 g, 72%) LCMS Rt=2.31 nm, [M+H]⁺=316.9-318.9.

### Synthesis of 2-(7-bromo-4-chloro-1-ethyl-1H-imidazo[4,5-c]pyridin-2-yl)acetonitrile (VII)

To N-(5-bromo-2-chloro-4-(ethylamino)pyridin-3-yl)-2-cyanoacetamide (3.15 g, 9.9 mmol) was added glacial acetic acid (35 ml). The suspension was stirred at reflux for 3 hours. The resulting solution was cooled to room temperature to yield the desired product which was used in the next step in situ. LCMS Rt=2.93 nm [M+H]⁺=298.8-300.8.

### Synthesis of 7-bromo-4-chloro-1-ethyl-N-hydroxy-1H-imidazo[4,5-c]pyridine-2-carbimidoyl cyanide (VIII)

To the solution of 2-(7-bromo-4-chloro-1-ethyl-1H-imidazo[4,5-c]pyridin-2-yl)acetonitrile was added slowly NaNO₂ (1.7 g, 24.8 mmol). The reaction was then stirred at room temperature overnight. The resulting solid was filtered, washed with water, and dried in a vacuum oven at 40° C. to yield the desired compound (3.16 g, 97% from VI). LCMS Rt=3.58 nm [M+H]⁺=327.8-329.8.

### Synthesis of 4-(7-bromo-4-chloro-1-ethyl-1H-imidazo[4,5-c]pyridin-2-yl)-1,2,5-oxadiazol-3-amine (IX)

To a stirred mixture of 7-bromo-4-chloro-1-ethyl-N-hydroxy-1H-imidazo[4,5-c]pyridine-2-carbimidoyl cyanide (3.12 g, 9.5 mmol) in dioxane (30 ml) and triethylamine (5.72 ml, 41 mmol) was added a solution of hydroxylamine (50% in water, 1.5 ml). The reaction was stirred at reflux for 6 hours. The reaction was then cooled to room temperature. The mixture was filtered and the filtrate evaporated to give a yellow solid. The solid was suspended in methanol (12 ml), warmed and stirred at 65° C. for 30 nm, then filtered to yield the desired product as a yellow solid (1.82 g, 56%) LCMS Rt=4.15 nm [M+H]⁺=343-347.

### Synthesis of 2-(4-amino-1,2,5-oxadiazol-3-yl)-4-chloro-1-ethyl-1H-imidazo[4,5-c]pyridin-7-ol (X)

To a suspension of 4-(7-bromo-4-chloro-1-ethyl-1H-imidazo[4,5-c]pyridin-2-yl)-1,2,5-oxadiazol-3-amine (1.82 g, 5.3 mmol) in tetrahyfrofuran (46 ml) at −78° C. was slowly added isopropylmagnesium chloride (2M in THF, 8.5 ml, 16.9 mmol). The temperature was kept below −70° C. during the addition. After stirring 10 nm at −78° C., trimethyl borate (2.12 ml, 18.5 mmol) was added and the reaction then stirred at −78° C. for 1 hour. The mixture was then warmed to room temperature and stirred overnight. The reaction was cooled at 0° C. Hydrogen peroxide (50% in water, 5.46 ml) and sodium hydroxide 3N (3.64 ml) were added together, keeping the temperature of the reaction below 40° C. The resulting mixture was stirred vigorously at room temperature for 2 hours. The organic solvent was removed under vacuum (water left) and the resulting suspension was acidified to pH=1 by addition of 1N hydrochloric acid. The mixture was stirred at room temperature for 30 nm. 45 ml of ethyl acetate were added and the reaction was stirred at room temperature a further 1 hour. The mixture was filtered (2 crops). The solid was washed with 9 ml of water, 9 ml of ethyl acetate, 9 ml of toluene, and 9 ml of ethyl acetate. After drying in a vacuum oven at 40° C., the desired compound was obtained as a yellow solid (1.31 m, 89%). LCMS Rt=3.26 nm, [M+H]⁺=281-283.

### Synthesis of tert-butyl 4-((2-(4-amino-1,2,5-oxadiazol-3-yl)-4-chloro-1-ethyl-1H-imidazo[4,5-c]pyridin-7-yloxy)methyl)piperidine-1-carboxylate (XI)

To a solution of 2-(4-amino-1,2,5-oxadiazol-3-yl)-4-chloro-1-ethyl-1H-imidazo[4,5-c]pyridin-7-ol (0.5 g, 1.78 mmol) in dimethylformamide (15 ml) was added cesium carbonate (1.45 g, 4.45 mmol) followed by tert-butyl 4-(bromomethyl)piperidine-1-carboxylate (0.545 g, 1.96 mmol). The reaction was stirred at 40° C. for 20 hours. The reaction was then partitioned between ethyl acetate and water. The aqueous layer was extracted twice with ethyl acetate. The combined organic layers were washed with brine and dried over MgSO4. The solvent was removed to give a yellow solid which was triturated with hexane/ethyl acetate to give the desired compound after filtation (0.497 g, 58%). LCMS Rt=4.97 nm, only fragments available.

### Synthesis of tert-butyl 4-((2-(4-amino-1,2,5-oxadiazol-3-yl)-1-ethyl-4-(3-hydroxy-3-methylbut-1-ynyl)-1H-imidazo[4,5-c]pyridin-7-yloxy)methyl)piperidine-1-carboxylate (XII)

In a microwave tube, tert-butyl 4-((2-(4-amino-1,2,5-oxadiazol-3-yl)-4-chloro-1-ethyl-1H-imidazo[4,5-c]pyridin-7-yloxy)methyl)piperidine-1-carboxylate (47 mg, 01 mmol), Zinc dust (0.002 g, 0.0033 mmol), Sodium iodide (0.005 g, 0.003 mmol), DBU (0.023 ml, 0.15 mmol), triethylamine (0.017 ml, 0.125 mmol), 2-methyl-3-butyn-2-ol (0.023 ml, 0.24 mmol), tetrakis triphenylphosphine palladium (0) (10% weight, 0.005 g) and DMSO (0.6 ml) were mixed together. The solution was degazed, then the tube was sealed and the reaction heated in a microwave at 80° C. for 2.5 hours. The reaction was diluted with ethyl acetate, washed with water. The aqueous layer was extracted twice with ethyl acetate. The combined organic layers were washed with water, brine, then dried over MgSO₄. The solvent was removed to give a crude product which was purified by prep HPLC to yield the desired compound (0.012 g, 23%) LCMS Rt=4.43 nm, only fragments available.

### Synthesis of 4-(2-(4-amino-1,2,5-oxadiazol-3-yl)-1-ethyl-7-(piperidin-4-ylmethoxy)-1H-imidazo[4,5-c]pyridin-4-yl)-2-methylbut-3-yn-2-ol hydrochloride salt (XIII)

tert-butyl 4-((2-(4-amino-1,2,5-oxadiazol-3-yl)-1-ethyl-4-(3-hydroxy-3-methylbut-1-ynyl)-1H-imidazo[4,5-c]pyridin-7-yloxy)methyl)piperidine-1-carboxylate (12 mg, 0.023 mmol) was dissolved in methanol (3 ml). HCl 4N in dioxane (2 ml) was added and the reaction was stirred at room temperature 2 hours. The solvent was removed under vacuum. The resulting solid was triturated in methanol (1.5 ml), filtered, rinsed with cold methanol (0.5 ml) to yield the desired compound as a white solid (9.7 mg, 92%). LCMS Rt=2.18 nm. Only fragments available. ¹H NMR (d6-DMSO, 400 MHz) δ=8.96 (s, br, 2H), 8.23 (s, 1H), 7.05 (s, br, 2H), 4.83 (q, 2H), 4.28 (d, 2H), 3.35 (m, 2H), 2.96 (m, 2H), 2.21 (s, br, 1H), 1.97 (d, 2H), 1.68 (m, 2H), 1.56 (s, 6H), 1.47 (t, 3H).

### Synthesis of 4-(4-chloro-1-ethyl-7-(piperidin-4-ylmethoxy)-1H-imidazo[4,5-c]pyridin-2-yl)-1,2,5-oxadiazol-3-amine formate salt (XIV)

To a solution of tert-butyl 4-((2-(4-amino-1,2,5-oxadiazol-3-yl)-4-chloro-1-ethyl-1H-imidazo[4,5-c]pyridin-7-yloxy)methyl)piperidine-1-carboxylate (0.045 g, 0.094 mmol) in methanol (4 ml) was added HCl 4N in dioxane (2 ml) and the reaction was stirred at room temperature for 3 hours. The solvent was removed and the residue triturated with methanol (2.5 ml). The residue was filtered then purified by prep HPLC to yield the desired product as a white powder (0.0185 g, 46%). LCMS Rt=2.28 nm. Only fragments available. ¹H NMR (d6-DMSO, 400 MHz) δ=8.40 (s, 1H), 8.01 (s, 1H), 6.96 (s, 2H), 4.84 (q, 21-1), 4.19 (d, 2H), 3.24 (m, 2H), 2.79 (m, 2H), 2.12 (s, br, 1H), 1.89 (m, 2H), 1.6-1.3 (m+t, 5H).

TABLE 1
Abbreviations

| | |
|---|---|
| Br₂ | Bromine |
| H₂O | Water |
| POCl₃ | Phosphorus oxychloride |
| HCl | Hydrochloric acid |
| SnCl₂ | Tin chloride |
| EDC | 1-ethyl-3-(3-dimethylaminopropyl) carbodiimide |
| | hydrochloride |
| DCM | Dichloromethane |
| NaNO₂ | Sodium nitrite |
| Et₃N | Triethylamine |
| B(OMe)₃ | Trimethoxyborate |
| H₂O₂ | Hydrogen peroxide |
| CsCO₃ | Cesium carbonate |
| DMF | Dimethyl formamide |
| THF | Tetrahydrofuran |
| Zn | Zinc |
| NaI | Sodium iodide |
| DBU | 1,8-Diazabicyclo[5.4.0]undec-7-ene |
| DMSO | Dimethyl sulfoxide |
| MeOH | Methanol |
| MgSO₄ | Magnesium sulfate |
| LCMS | Liquid chromatography Mass Spectroscopy |
| Rt | Retention time |
| NMR | Nuclear Magnetic resonance |
| MHz | Megahertz |
| s | Singlet |
| d | Doublet |
| t | Triplet |
| q | Quadruplet |
| m | Multiplet |
| H | Hydrogen |
| br | Broad |
| MW | Molecular weight |
| V | Volt |
| amu | Atomic mass unit |
| ml | Millilitre |
| min | Minute |
| mmol | Millimole |
| g | Gram |
| API | Atomic pressure ionisation |
| ES | Electrospray |

### NMR, LCMS and Prep HPLC Conditions:

All reactions were carried out under inert atmosphere.
NMR spectra were obtained on a Bruker dpx400.
The LCMS analytical samples were run on an Agilent HP1100-LC/MSD VL system using the following conditions:

Column: Phenomenex Gemini C18 30×3 mm 3 μm

Solvents: A=Water+0.1% Formic acid; B=Acetonitrile+0.1% Formic acid

Flow Rate:1.2 ml/min

Temperature: 40° C.

TABLE 2
Gradient conditions

| Time (min) | % A | % B |
|---|---|---|
| 0.00 | 95.0 | 5.0 |
| 6.00 | 5.0 | 95.0 |
| 7.50 | 5.0 | 95.0 |
| 7.60 | 95.0 | 5.0 |
| 8.00 | 95.0 | 5.0 |

Wavelength:

254 nm (reference at 400 nm)

210 nm (reference at 360 nm)

Mass spectrometry conditions:

The mass spec data were gathered in positive mode, scanning for masses between 150 and 700 amu, using a fragmentor ramp from 118V to 400V (for MW=118.09 to MW=922.01 respectively). The prepared samples were run on a Waters-ZQ prep system using the following conditions:

Column: Phenomenex Gemini C18 100×21.20 mm 5 μm

Solvents: A=Water+0.1% Formic acid; B=(95% Acetonitrile: 5% Water)+0.1% Formic acid

Flow Rate: 20 ml/min

Temperature: Room temperature

Gradient conditions: The gradient conditions were variable depending on the retention time of each compound

Wavelength: PDA detection from 190-600 nm

Mass spec conditions:

The mass spec data were gathered in positive and negative mode, from 150 to 700 amu, using API and ES modes.

### Immobilization on Beads (Immobilization Product; Affinity Matrix)

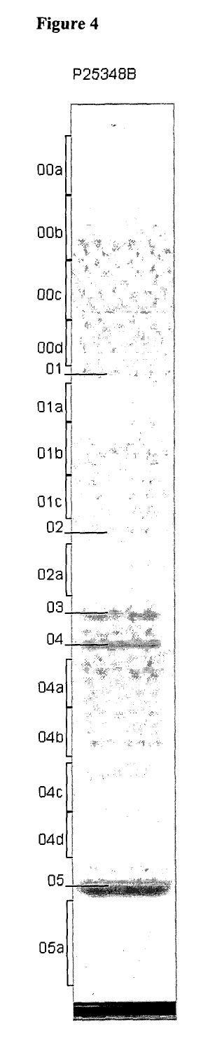

NHS-activated Sepharose 4 Fast Flow (Amersham Biosciences, 17-0906-01) was equilibrated with anhydrous DMSO (Dimethylsulfoxid, Fluka, 41648, H20<=0.005%). 1 ml of settled beads was placed in a 15 ml Falcon tube, compound stock solution (mixture of two isomers as shown in FIG. 4; usually 100 mM in DMF or DMSO) was added (final concentration 0.2-2 μmol/ml beads) as well as 15 μl of triethylamine (Sigma, T-0886, 99% pure). Beads were incubated at room temperature in darkness on an end-over-end shaker (Roto Shake Genie, Scientific Industries Inc.) for 16-20 hours. Coupling efficiency is determined by HPLC. Non-reacted NHS-groups were blocked by incubation with aminoethanol at room temperature on the end-over-end shaker over night. Beads were washed with 10 ml of DMSO and were stored in isopropanol at −20° C. These beads were used as the affinity matrix in example 2 and 3. Control beads (no ligand immobilized) were generated by blocking the NHS-groups by incubation with aminoethanol as described above.

### Example 2

### Drug Pulldown of PARP Proteins Using Immobilized Compounds

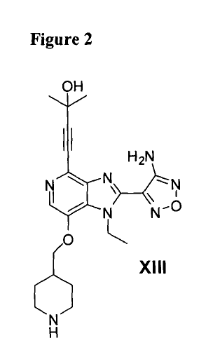

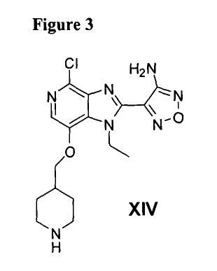

This example demonstrates the use of the immobilized compounds (see FIGS. 2 and 3) for the capturing and identification of the PARP proteins from mixed Jurkat and Ramos cell lysate. To this end, a mixture of lysates of Jurkat and Ramos cells was contacted with the immobilization product (affinity matrix) described in example 1. Proteins bound to the immobilized compounds were identified by mass spectrometry (MS) analysis. Further experimental protocols can be found in WO2006/134056.

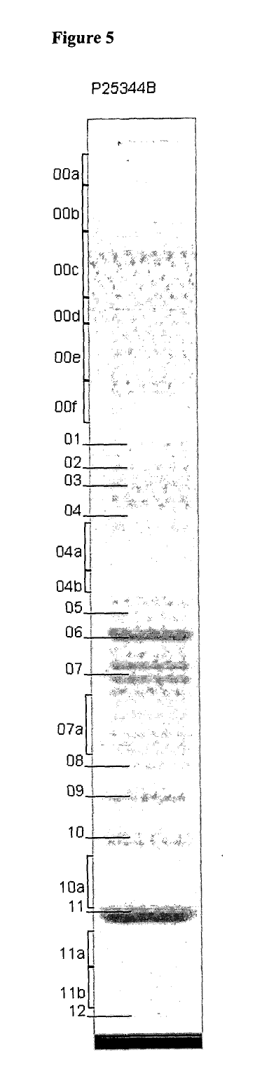

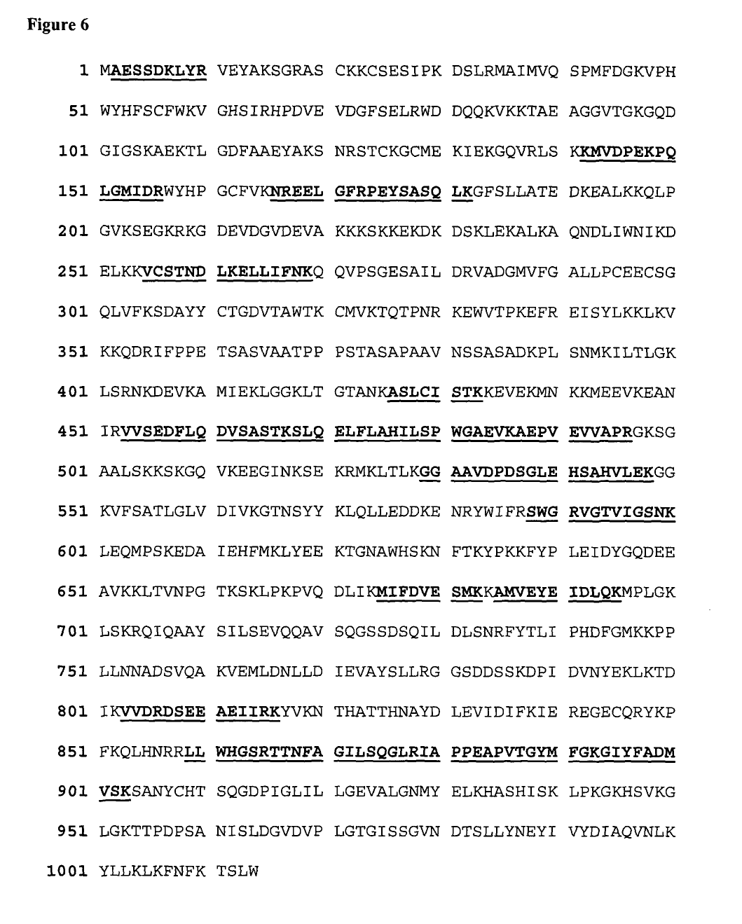

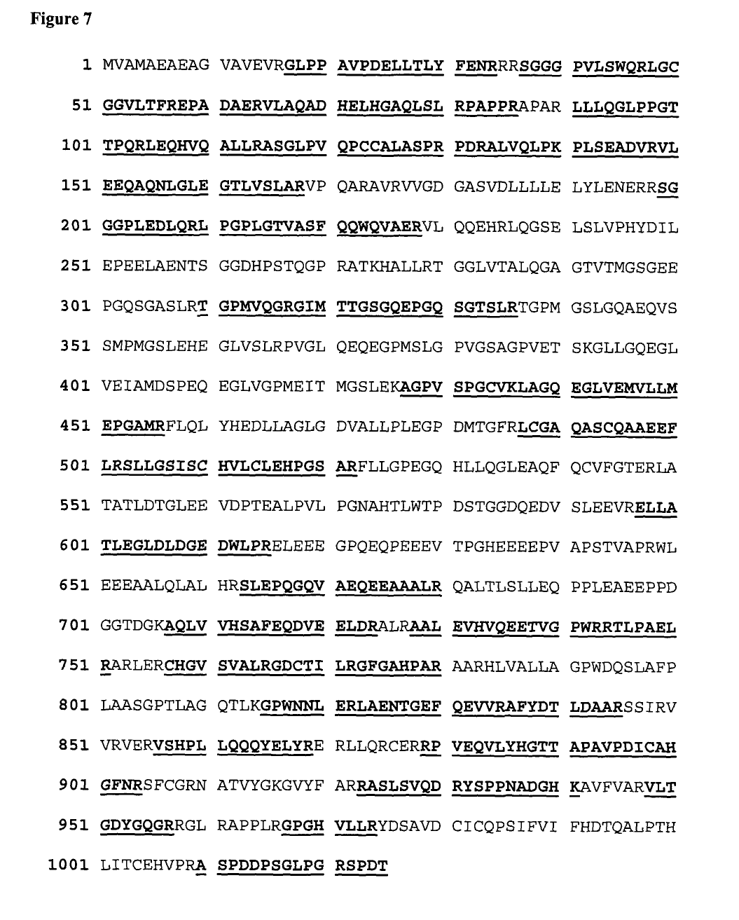

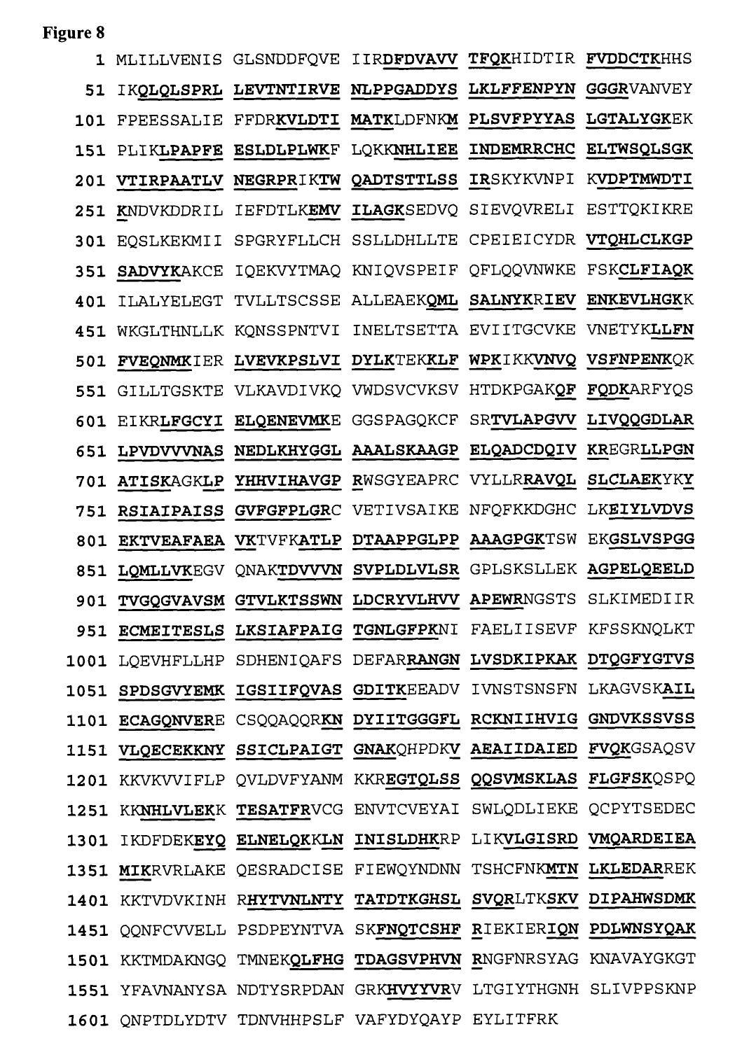

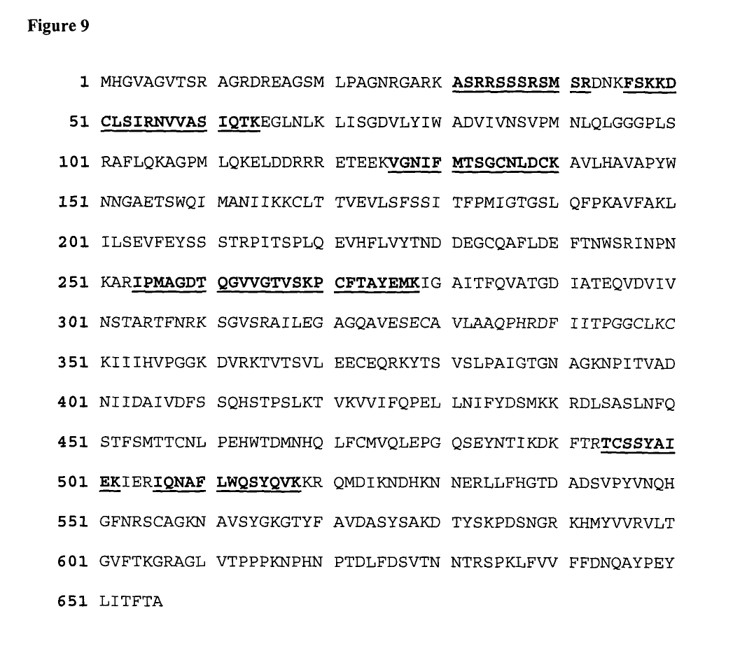

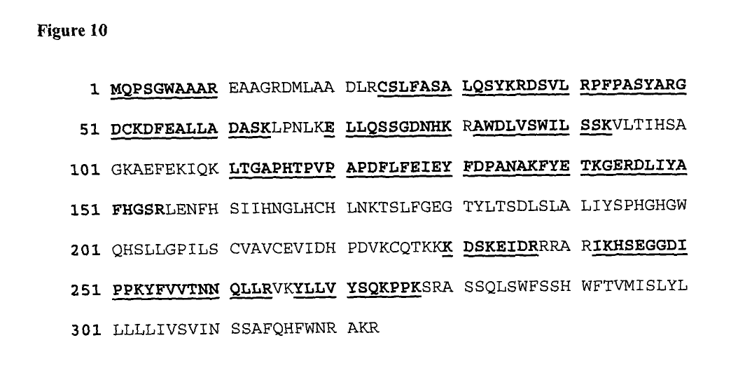

For the identification of proteins by mass spectrometry analysis the proteins captured by the affinity matrix were eluted in SDS sample buffer and subsequently separated by SDS-Polyacrylamide gel elecrophoresis (FIGS. 4 and 5). Suitable gel bands were cut out and subjected to in-gel proteolytic digestion with trypsin and analyzed by LC-MS/MS mass spectrometry. The identification of PARP protein derived peptides by mass spectrometry is documented in table 4 and the peptide sequence coverage of the PARP protein sequences is shown in FIGS. 6 to 10.

### 1. Cell Culture

Jurkat cells (ATCC number TIB-152) and Ramos cells (ATCC number CRL-1596) were either obtained from an external supplier (CIL SA, Mons, Belgium) or grown in one litre Spinner flasks (Integra Biosciences, #182101) in suspension in RPMI 1640 medium (Invitrogen, #21875-034) supplemented with 10% Fetal Bovine Serum (Invitrogen, #10270-106) at a density between 0.2×10⁶ and 1.0×10⁶ cells/ml. Cells were harvested by centrifugation, washed once with 1×PBS buffer (Invitrogen, #14190-094) and cell pellets were frozen in liquid nitrogen and subsequently stored at −80° C.

### 2. Preparation of Cell Lysates

Cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1 873 580) per 25 ml buffer was added. The material was dounced 20 times using a mechanized POTTER S, transferred to 50 ml falcon tubes, incubated for 30 minutes on ice and spun down for 10 minutes at 20,000×g at 4° C. (10,000 rpm in Sorvall SLA600, precooled). The supernatant was transferred to an ultracentrifuge (UZ)-polycarbonate tube (Beckmann, 355654) and spun for 1 hour at 160.000×g at 4° C. (42.000 rpm in Ti50.2, precooled). The supernatant was transferred again to a fresh 50 ml falcon tube, the protein concentration was determined by a Bradford assay (BioRad) and samples containing 50 mg of protein per aliquot were prepared. The samples were immediately used for experiments or frozen in liquid nitrogen and stored frozen at −80° C. This procedure was applied for the preparation of Ramos and Jurkat cell lysates.

### 3. Drug Pull-Down Experiment

Sepharose-beads with the immobilized ligand (100 μl beads per pull-down experiment) were equilibrated in lysis buffer and incubated with a cell lysate sample containing 50 mg of protein on an end-over-end shaker (Roto Shake Genie, Scientific Industries Inc.) for 2 hours at 4° C. Beads were collected, transfered to Mobicol-columns (MoBiTech 10055) and washed with 10 ml lysis buffer containing 0.4% NP40 detergent, followed by 5 ml lysis buffer with 0.2% detergent. To elute the bound protein, 60 μl 2×SDS sample buffer was added, the column was heated for 30 minutes at 50° C. and the eluate was transferred to a to microfuge tube by centrifugation. Proteins were then alkylated with 108 mM iodoacetamid. Proteins were then separated by SDS-Polyacrylamide electrophoresis (SDS-PAGE).

### 4. Protein Identification by Mass Spectrometry

### 4.1 Protein Digestion Prior to Mass Spectrometric Analysis

Gel-separated proteins were reduced and digested in gel essentially following the procedure described by Shevchenko et al., 1996, Anal. Chem. 68:850-858. Briefly, gel-separated proteins were excised from the gel using a clean scalpel, reduced using 10 mM DTT (in 5 mM ammonium bicarbonate, 54° C., 45 minutes) at room temperature in the dark. The reduced proteins were digested in gel with porcine trypsin (Promega) at a protease concentration of 12.5 ng/μl in 5 mM ammonium bicarbonate. Digestion was allowed to proceed for 4 hours at 37° C. and the reaction was subsequently stopped using 5 μl 5% formic acid.

### 4.2 Sample Preparation Prior to Analysis by Mass Spectrometry

Gel plugs were extracted twice with 20 μl 1% TFA and pooled with acidified digest supernatants. Samples were dried in a a vaccuum centrifuge and resuspended in 10 μl 0.1% formic acid.

### 4.3. Mass Spectrometric Data Acquisition

Peptide samples were injected into a nano LC system (CapLC, Waters or Ultimate, Dionex) which was directly coupled either to a quadrupole TOF (QTOF2, QTOF Ultima, QTOF Micro, Micromass) or ion trap (LCQ Deca XP) mass spectrometer. Peptides were separated on the LC system using a gradient of aqueous and organic solvents (see below). Solvent A was 5% acetonitrile in 0.5% formic acid and solvent B was 70% acetonitrile in 0.5% formic acid.

TABLE 3
Peptides eluting off the LC system were partially sequenced
within the mass spectrometer

| Time (min) | % solvent A | % solvent B |
|---|---|---|
| 0 | 95 | 5 |
| 5.33 | 92 | 8 |
| 35 | 50 | 50 |
| 36 | 20 | 80 |
| 40 | 20 | 80 |
| 41 | 95 | 5 |
| 50 | 95 | 5 |

### 4.4. Protein Identification

The peptide mass and fragmentation data generated in the LC-MS/MS experiments were used to query fasta formatted protein and nucleotide sequence databases maintained and updated regularly at the NCBI (for the NCBInr, dbEST and the human and mouse genomes) and European Bioinformatics Institute (EBI, for the human, mouse, D. to melanogaster and C. elegans proteome databases). Proteins were identified by correlating the measured peptide mass and fragmentation data with the same data computed from the entries in the database using the software tool Mascot (Matrix Science; Perkins et al., 1999. Probability-based protein identification by searching sequence databases using mass spectrometry data. Electrophoresis 20, 3551-3567). Search criteria varied depending on which mass spectrometer was used for the analysis. Sequence identifiers are defined by the International Protein Index (IPI) (Kersey et al., 2004. Proteomics 4(7): 1985-1988).

TABLE 4
Identification of PARP proteins by mass spectrometry
in drug pulldown experiments with immobilized compounds
XIII and XIV

| PARP protein | Compound XIII | Compound XIV |
|---|---|---|
| PARP1 | + | + |
| PARP10 | + | + |
| PARP14 | + | + |
| PARP15 | + | − |
| PARP16 | + | + |

1. An immobilization compound of formula (I) 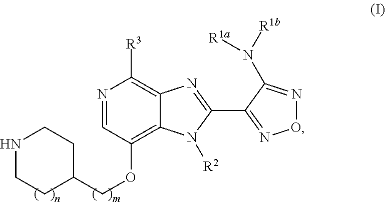
or a salt thereof, wherein
R^{1a}, R^{1b}, R² are independently selected from the group consisting of H; and C₁₋₄ alkyl, wherein C₁₋₄ alkyl is optionally substituted with one or more halogen, which are the same or different;
R³ is H; halogen; CN; C(O)OR⁴; OR⁴; C(O)R⁴; C(O)^{N}(R⁴R^{4a}); S(O)₂N(R⁴R^{4a}); S(O)N(R⁴R^{4a}); S(O)₂R⁴; S(O)R⁴; SR⁴; N(R⁴R^{4a}); NO₂; OC(O)R⁴; N(R⁴)C(O)R^{4a}; N(R⁴)S(O)₂R^{4a}; N(R⁴)S(O)R^{4a}; N(R⁴)C(O)N(R^{4a}R^{4b}); N(R⁴)C(O)OR^{4a}; OC(O)N(R⁴R^{4a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R⁵, which are the same or different;
R⁴, R^{4a}, R^{4b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R⁵, which are the same or different;
R⁵ is halogen; CN; OR⁶; SR⁶; N(R⁶R^{6a}); or NO₂;
R⁶, R^{6a} are independently selected from the group consisting of H; and C₁₋₄ alkyl, wherein C₁₋₄ alkyl is optionally substituted with one or more halogen, which are the same or different;
m is 0; 1; or 2;
n is 0; 1; or 2.

2. The immobilization compound of claim 1, selected from the group consisting of

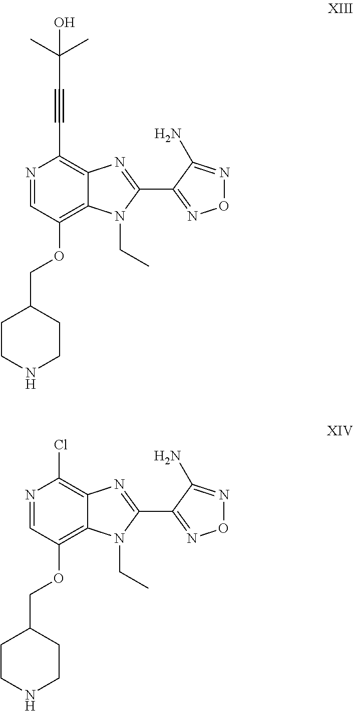

3. The immobilization compound according to claim 1 wherein the immobilization compound is immobilized on a solid support.

4. The immobilization compound of claim 3, wherein the immobilization compound is covalently bound by direct or linker mediated attachment the immobilization compound to the solid support, wherein the linker is a C₁₋₁₀ alkylene group, which is optionally interrupted by one or more atoms or functional groups selected from the group consisting of S, O, NH, C(O)O, C(O), and C(O)NH and wherein the linker is optionally substituted with one or more substituents independently selected from the group consisting of halogen, OH, NH₂, C(O)H, C(O)NH₂, SO₃H, NO₂, and CN.

5. (canceled)

6. A method for the identification of a PARP interacting compound, comprising the steps of
a) providing a protein preparation containing PARP,
b) contacting the protein preparation with the immobilization compound of claim 3 and with a given compound under conditions allowing the formation of a complex between PARP and the immobilization product, and
c) detecting the complex formed in step b).

7. A method for the identification of a PARP interacting compound, comprising the steps of:
a) providing two aliquots of a protein preparation containing PARP,
b) contacting one aliquot with the immobilization compound of claim 3 under conditions allowing the formation of a complex between PARP and the immobilization product,
c) contacting the other aliquot with the immobilization product and with a given compound under conditions allowing the formation of the complex, and
d) determining the amount of the complex formed in steps b) and c).

8. A method for the identification of a PARP interacting compound, comprising the steps of:
a) providing two aliquots comprising each at least one cell containing PARP,
b) incubating one aliquot with a given compound,
c) harvesting the cells of each aliquot,
d) lysing the cells in order to obtain protein preparations,
e) contacting the protein preparations with the immobilization compound of claim 3 under conditions allowing the formation of a complex between PARP and the immobilization product, and
f) determining the amount of the complex formed in each aliquot in step e).

9. The method of claim 7, wherein a reduced amount of the complex formed in the aliquot incubated with the compound in comparison to the aliquot not incubated with the compound indicates that PARP is a target of the compound.

10. The method of claim 7, wherein the amount of the complex is determined by separating PARP from the immobilization product and subsequent detection of separated PARP or subsequent determination of the amount of separated PARP, in particular wherein PARP is detected or the amount of PARP is determined by mass spectrometry or immunodetection methods, preferably with an antibody directed against PARP.

11. The method of claim 7, wherein said given compound is selected from the group consisting of synthetic compounds, or organic synthetic drugs, more preferably small molecule organic drugs, and natural small molecule compounds.

12. The method of claim 7, wherein the given compound is a PARP inhibitor.

13. A method for the purification of PARP, comprising the steps of
a) providing a protein preparation containing PARP,
b) contacting the protein preparation with the immobilization compound of claim 3 under conditions allowing the formation of a complex between PARP and the immobilization product, and
c) separating PARP from the immobilization product.

14. The method of claim 7, wherein the provision of a protein preparation includes the steps of harvesting at least one cell containing PARP and lysing the cell.

15. The method of claim 7, wherein the steps of the formation of the complex are performed under essentially physiological conditions.

16. A method for determining the presence of PARP in a sample, comprising the steps of:
a) providing a protein preparation expected to contain PARP,
b) contacting the protein preparation with the immobilization product compound of claim 3 under conditions allowing the formation of a complex between PARP and the immobilization product, and
detecting whether PARP has formed a complex with the immobilization product.

17. (canceled)

18. The immobilization compound of claim 3 wherein the solid support is selected from the group consisting of agarose, modified agarose, sepharose beads, latex, cellulose, and ferro- or ferrimagnetic particles.

19. The immobilization compound of claim 4 wherein said immobilization occurs via the ring nitrogen atom of the saturated ring in formula (I).

20. The method of claim 8, wherein a reduced amount of the complex formed in the aliquot incubated with the compound in comparison to the aliquot not incubated with the compound indicates that PARP is a target of the compound.

21. The method of claim 8, wherein the amount of the complex is determined by separating PARP from the immobilization product and subsequent detection of separated PARP or subsequent determination of the amount of separated PARP, in particular wherein PARP is detected or the amount of PARP is determined by mass spectrometry or immunodetection methods, preferably with an antibody directed against PARP.

22. The method of claim 8, wherein said given compound is selected from the group consisting of synthetic compounds, or organic synthetic drugs, more preferably small molecule organic drugs, and natural small molecule compounds.

23. The method of claim 8, wherein the given compound is a PARP inhibitor.

24. The method of claim 8, wherein the steps of the formation of the complex are performed under essentially physiological conditions.
